(19) United States
(12) Patent Application Publication
(10) Pub. No.: US 2004/0225450 A1
(43) Pub. Date: Nov. 11, 2004

(54) Analysis of substrates exposed to tobacco and components derived from tobacco

(76) Inventors: Michael W. Ogden, Pfafftown, NC (US); David W. Bombick, Winston-Salem, NC (US)

Correspondence Address: COLLIER SHANNON SCOTT, PLLC, 3050 K STREET, NW, SUITE 400, WASHINGTON, DC, 20007, US

(21) Appl. No.: 10/434,232

(22) Filed: May 9, 2003

(51) Int. Cl. G06F 19/00; G01N 33/48; G01N 33/50; G01N 31/00

(52) U.S. Cl. 702/22

(57) ABSTRACT

An innovative analytical method comprising the step of exposing a first substrate to at least one tobacco component, a tobacco composition, or at least one component derived from tobacco, such as, but not limited to, at least one tobacco smoke component. An additional step may comprise the step of preparing a second substrate not exposed to the tobacco component in an identical manner, wherein the second substrate may receive a different exposure, no exposure, or exposure in combination with another factor or condition, or any combination thereof. Additional steps may comprise collecting at least one first sample from the first substrate and at least one second sample from the second substrate, and analyzing the first and second sample by using at least one analytical characterization technique, wherein the analytical characterization technique comprises NMR spectroscopy. An additional step may comprise comparing the results of the analysis of the first and second samples using data pattern recognition techniques or metabonomic technology.

### FIELD OF THE INVENTION

The present invention relates to methods for the analysis of substrates exposed to a stimulus. More particularly, embodiments of the present invention relate to methods for the analysis of substrates and samples exposed to tobacco and compositions derived from tobacco, including tobacco components and tobacco smoke components.

### BACKGROUND OF THE INVENTION

Tobacco is one of the more complex plants known, drawing it's complexity from both genetic and environmental sources. Of the more than 60 genetic species of plants belonging to the genus Nicotiana, only two (N. Tabacum and N. Rustica) are widely cultivated for use as tobacco. The tobacco of commerce, N. Tabacum, is a hybrid plant capable of a high degree of variability as evidenced by the different major commercial types (flue-cured, burley, Maryland, and Oriental) and the numerous agronomic varieties within each type. Each of these types, in various combinations, and in addition to others, is currently used in the manufacture of cigarettes, cigars, and pipe tobaccos. See Ogden and Nelson, Modern Methods of Plant Analysis, Vol. 15, pp. 163-189 (1994); and Davis and Nielson, Tobacco Production, Chemistry & Technology (1999).

Superimposed on the composition of the tobacco leaf constituents is a mixture of enormous complexity resulting from combustion of these tobaccos on smoking. A 1982 review estimates that at least 3,875 individual components had been identified to date in tobacco smoke. See Dube and Green, Recent Advances in Tobacco Science, Vol. 8, pp. 42-102 (1982). By 1987, the number of tobacco smoke components reported in the literature had increased to over 4,500. See Chemical and Biological Studies on New Cigarette Prototypes that Heat Instead of Burn Tobacco, R. J. Reynolds Tobacco Company, p. 121 (1988).

The smoke from burning tobacco is typically characterized as either mainstream smoke, sidestream smoke, or environmental tobacco smoke. Mainstream smoke is that smoke emerging through the mouth-end of the tobacco product during puffing and is primarily inhaled by the smoker. Sidestream smoke is principally that smoke emanating from the burning end of the tobacco product. Environmental tobacco smoke is a dynamic mixture of substances present in ambient air as a specific result of tobacco smoking, and consists primarily of aged and diluted sidestream smoke and aged and diluted exhaled mainstream smoke. Technology and analytical methods developed over the years in tobacco and tobacco smoke studies are used in concert with more recently developed techniques to examine the chemical characteristics of smoke from current market products and research cigarettes, including new product prototypes and experimental cigarettes. See Green and Rodgman, Recent Advances in Tobacco Science, Vol. 22, pp. 131-304 (1996).

In general, laboratory testing of cigarette mainstream and sidestream smoke and smoke yield requires smoke generation and trapping on a smoking machine operated under standardized or accelerated smoking regimens. See Tobacco Production, Chemistry and Technology, Baker, p. 398 (1999); Borgerding et al., Journal of the Association of Official Analytical Chemists, Vol. 73(4), pp. 605-609 (1990); Borgerding and Winkler, CORESTA Information Bulletin Vol. 1995; Podraza et al., 1999 Massachusetts Benchmark Study to Evaluate Mainstream and Sidestream Cigarette Smoke Constituent Yields. Part I. General Summary and Overview of Results, 54^{th} TSRC (2000); Taylor et al., 1999 Massachusetts Benchmark Study to Evaluate Mainstream and Sidestream Cigarette Smoke Constituent Yields. Part II. Overview of Mainstream Smoke Results, 54^{th} TSRC (2000). For recent examples of cigarette testing and comparisons, See deBethizy et al., Journal of Clinical Pharmacology, Vol. 30(8), pp. 755-763 (1990); Borgerding et al., Food and Chemical Toxicology, Vol. 36(7), pp. 169-182 (1997); and Swauger et al., Regulatory Toxicology and Pharmacology, Vol. 35(2 Part 1), pp142-156 (2002).

Specific chemical testing of tobacco and tobacco components, including tobacco smoke, generally relies on the established analytical techniques of gas chromatography (GC) and GC coupled with mass spectrometry (MS), liquid chromatography (LC) and LC coupled with MS, and by ion chromatography. See Ogden, Journal of High Resolution Chromatography, Vol. 11, p 341 (1988); Byrd et al., Journal of Chromatography, Vol. 503(2), pp 359-3368 (1990); White et al., Journal of Chromatographic Science, Vol. 28(8), pp 393-399 (1990); Nanni et al., Journal of Chromatography, Vol. 505(2), pp365-374 (1990); Chung et al., Journal of Liquid Chromatography, Vol. 6, pp 425-444 (1983); Risner and Cash, Journal of Chromatographic Science, Vol. 28(5), pp 239-244 (1990); Risner, Beiträge zur Tabakforschung International, Vol. 15, pp 11-17 (1991); Risner, Journal of Chromatographic Science, Vol. 32, pp 76-82 (1993); Nanni et al., Journal of Chromatographic Science, Vol. 28(8), pp 432-436 (1990). General screening techniques also include use of chromatographic profiling. See Rogers et al., Journal of Chromatographic Science, Vol. 35(5), pp. 193-200 (1997). More specialized infrared spectroscopic techniques also have been used, although generally not routinely, either in combination with chromatographic separation, or on their own. See Coleman and Gordon, Journal of Chromatographic Science, Vol. 29(9), pp 371-376 (1991); Cole and Martin, Analyst, Vol. 121(April), pp 495-500 (1996).

There are a number of diseases and pathologies that have been reported to be associated with tobacco use. See Hoffmann and Harris, Mechanisms in Tobacco Carcinogenesis, Banbury Report 23, Cold Spring Harbor Laboratory, Cold Spring Harbor, N.Y. (1986); Crystal and West, Lung Injury, Raven Press, New York, N.Y. (1991); Parent, Comparative Biology of the Normal Lung, CRC Press, Boca Raton, Fla. (1991); Wang, Carbon Monoxide and Cardiovascular Functions, CRC Press, Boca Raton, Fla. (2001); Diana, Tobacco Smoking and Atherosclerosis: Pathogens and Cellular Mechanisms. Advances in Experimental Medicine and Biology, Vol. 273, Plenum Press, New York (1990); Clayson, Toxicological Carcinogenesis, Lewis Publishers, Boca Raton, Fla. (2001).

A number of in vitro, in vivo, and human biological tests have been used to assess the biological activity of tobacco smoke and tobacco smoke components. In vitro tests to assess tobacco smoke include measures of genetic damage (genotoxicity and clastogenicity), cell death (cytotoxicity), and cell physiological processes.

Genotoxicity tests include the Ames bacterial mutagenicity assay, the sister chromatid exchange (SCE) assay, the chromosomal aberration assay, and the micronuclei induction assay. The Ames bacterial mutagenicity assay has been used to determine the mutagenicity of cigarette smoke condensates from a representative sample of cigarettes in the US market, nicotine and its metabolites, glycerol used in cigarette manufacture, cigarette smoke condensates obtained at different pyrolysis temperatures, sidestream cigarette smoke, environmental tobacco smoke, and cigarette smoke condensates from certain types of cigarettes. See Steele et al., Mutation Research, Vol. 342, pp. 179-190 (1995); Chepiga et al., Food and Chemical Toxicology, Vol. 38, pp. 949-962 (2000); Doolittle et al., Mutation Research, Vol. 344, pp. 95-102 (1995); Doolittle et al., Food and Chemical Toxicology, Vol. 26, pp. 631-635 (1988); White et al., Food and Chemical Toxicology, Vol. 39, pp. 499-505 (2001); Doolittle et al., Mutation Research, Vol. 240, pp. 59-72 (1990); Bombick et al., Environmental and Molecular Mutagenesis, Vol. 31, pp. 169-175 (1998); Lee et al., Mutation Research, Vol. 242, pp. 37-45 (1990); Bombick et al., Fundamental and Applied Toxicology, Vol. 39, pp. 11-17 (1997); Bombick et al., Food and Chemical Toxicology, Vol. 36, pp. 183-190 (1998). Also, the Ames bacterial mutagenicity assay has been used to evaluate the inhibition of cigarette smoke condensate on the mutagenicity of heterocyclic amines, the inhibition of nitrosamine mutagenicity by tobacco smoke and its constituents, and the inhibition of tobacco smoke, nicotine, and cotinine on the mutagenicity of certain tobacco specific nitrosamines (TSNA), the inhibition of nitrosamine mutagenicity by tobacco smoke and its constituents, and the inhibition of tobacco smoke, nicotine, and cotinine on the mutagenicity of certain tobacco specific nitrosamines (TSNA). See Lee et al., Mutation Research, Vol. 322, p. 21-32 (1994); Lee et al., Mutation Research, Vol. 367, pp. 83-92 (1996); Brown et al., Mutation Research, Vol. 494, pp. 21-29 (2001). The SCE assay has examined clastogenicity in cells after exposure to oxygen radicals and exposure to cigarette smoke from certain types of cigarettes. See Lee et al., Environmental and Molecular Mutagenesis, Vol. 13, pp. 54-59 (1989); Lee et al., Mutation Research, Vol. 240, pp. 251-257j (1990); Bombick et al., Fundamental and Applied Toxicology, Vol. 39, pp. 11-17 (1997); Bombick et al., Toxicology In Vitro, Vol. 12, pp. 241-249 (1998).

Chromosomal aberrations have been examined in cells exposed to smoke condensates from certain types of cigarettes. See Lee et al., Mutation Research, Vol. 242, pp. 37-45 (1990); Bombick et al., Environmental and Molecular Mutagenesis, Vol. 31, pp. 169-175 (1998)]. The induction of micronuclei has been evaluated in cells isolated from bone marrow of animals exposed to cigarette smoke. See Lee et al., Mutation Research, Vol. 240, pp. 251-257 (1990); Coggins et al., Inhalation Toxicology, Vol. 2, pp. 407-431 (1990).

Cytotoxicity of cigarette smoke has been evaluated in a number of cytotoxicity assays. See Putnam et al., Toxicology In Vitro, Vol. 16, pp. 599-607 (2002). Cytotoxicity has been assessed in cigarette smoke condensates, sidestream smoke, whole mainstream smoke, and environmental tobacco smoke. See Bombick et al., Environmental and Molecular Mutagenesis, Vol. 17, p. 11 (1991); Bombick et al., 1997; Bombick et al., 1998). Cigarette smoke condensates from a representative sample of products from the US market have been evaluated for cytotoxicity. See Putnam et al., Toxicological Sciences, Vol. 60, p. 305 (2001). Cytotoxicity has been determined for a number of compounds found in cigarette smoke including nicotine, pyridines, aldehydes, and hydroxy- and dihydroxybenzenes. See Bombick and Doolittle, In Vitro Toxicology, Vol. 8, pp. 349-356 (1995); Bombick et al., 1998; Bombick et al., Toxicological Sciences, Vol. 48, p. 65 (1999). Tobacco smoke from certain types of cigarettes also has been evaluated for cytotoxic activity. See Bombick et al., 1997; Bombick et al., 1998; Bombick et al., 1998).

Other in vitro tests including gap junctional intercellular communication, indicators of plasma membrane integrity, replicative DNA synthesis, and measurement of intracellular oxidation have been developed to evaluate tobacco smoke. See McKarns and Doolittle, In Vitro Toxicology, Vol. 4, pp. 81-92 (1991); Bombick and Doolittle, Toxicology Methods, Vol. 2, pp. 255-264 (1992); Ayres et al., Inhalation Toxicology, Vol. 13(2), pp. 149-186 (1995). Some of these methods have been used to evaluate the biological activity of smoke condensates from different types of cigarettes. See McKarns et al., Toxicology In Vitro, Vol. 4, pp. 41-51 (2000).

Animal studies have also been used to examine the biological activity of tobacco smoke. Animals used in these studies include rats, mice, hamster, and dogs. See Coggins, Toxicologic Pathology, Vol. 29, pp. 550-57 (2001). Rats have been used to evaluate the toxicity of mainstream and sidestream smoke as well as an aged and diluted sidestream smoke that served as a surrogate for environmental tobacco smoke. See Baumgartner and Coggins, Beitrage zur Tabakforschung International, Vol. 10, pp. 169-174 (1980); Coggins et al., Inhalation Toxicology, Vol. 5(1), pp. 77-95 (1993). The carcinogenic potential of tobacco smoke condensates has been evaluated by a mouse skin painting assay. See Chemical and Biological Studies on New Cigarette Prototypes that Heat Instead of Burn Tobacco, R. J. Reynolds Tobacco Company, 1988; Meckley et al., The Toxicologist, No. 1085 (1998); Meckley et al., The Toxicologist, No. 1107 (1999). Rodent assays have been employed in the evaluation of certain types of cigarettes. See Coggins et al., Fundamental Applied Toxicology, Vol. 13, pp. 460-483 (1989); Coggins et al., Inhalation Toxicology, Vol. 1, pp. 197-226 (1989). In vivo studies have also been used to examine specific biological endpoints after exposure of the animals to tobacco smoke. These endpoints include genotoxicity endpoints as urine mutagenicity, DNA adduct formation, and micronuclei in bone marrow cells. See Doolittle et al., Mutation Research, Vol. 206, pp. 141-148 (1988); Doolittle et al., Mutation Research, Vol. 260, pp. 9-18 (1991); Lee et al., Fundamental & Applied Toxicology, Vol. 19, pp. 141-146 (1992); Brown et al., Experimental and Toxicologic Pathology, Vol. 47, pp. 183-191 (1995); Brown et al., Environmental and Molecular Mutagenesis, Vol. 29, pp. 303-311 (1997); Brown et al., Mutation Research, Vol. 414, pp. 21-30 (1998); Chang et al., Tobacco Science, Vol. 42, pp. 38-45 (1998); Brown et al., Toxicological Sciences, Vol. 47, pp. 33-39 (1999); Lee et al., Mutation Research, Vol. 240, pp. 251-257 (1990). Cell proliferation has been measured in rats exposed to cigarette smoke. See Ayres et al., Inhalation Toxicology, Vol. 7, pp. 1225-1246 (1995). Rodents also have been used as a model to investigate various aspects of nicotine metabolism. See deBethizy et al., Journal of Clinical Pharmacology, Vol. 30, pp. 755-763 (1990); Chang and deBethizy, The Toxicologist, Vol. 36(1), p. 30 (1997).

There have been a number of studies using human subjects to evaluate the biological activity of cigarette smoke. Many human studies have examined the behaviors associated with smoking. Specifically, behaviors including sensory attributes, psychophysiological parameters, and puff profiles during smoking have been examined. See Chemical and Biological Studies on New Cigarette Prototypes that Heat Instead of Burn Tobacco, R. J. Reynolds Tobacco Co any, 1988; Pritchard et al., Psychophysiology, Vol. 28 (1991); Pritchard et al., Psychopharmacology, Vol. 108(4), pp. 437-442 (1992); Robinson et al., Psychopharmacology, Vol. 108, pp. 466-472 (1992); Robinson et al., Psychopharmacology, Vol. 108, pp. 466-472 (1992); Pritchard et al., Psychophysiology, Vol. 32, pp. 19-27 (1995); Pritchard et al., Psychopharmacology, Vol. 128, p. 432 (1996); Pritchard et al., Psychopharmacology, Vol. 127, pp. 55-62 (1996); Pritchard et al., Neuropsychobiology, Vol. 34, pp. 208-221 (1996); Walker et al., Assessment of Possible Perceptual, Cognitive and Affective Effects of Sidestream Smoke on Non-Smokers, 1996 CORESTA Congress (1997); Pritchard et al., Psychopharmacology, Vol. 143(3), pp. 273-279 (1999); Stiles et al., The Toxicologist, Vol. 48, pp. 119-120 (1999). Additionally, several studies have examined nicotine uptake, nicotine pharmacokinetics, and nicotine metabolites in smokers using various biological fluids as saliva, blood, and urine. See Chemical and Biological Studies on New Cigarette Prototypes that Heat Instead of Burn Tobacco, R. J. Reynolds Tobacco Company, 1988; Robinson et al., The Toxicologist, Vol. 8, p. 205 (1988); deBethizy et al., Journal of Clinical Pharmacology, Vol. 30, pp. 755-763 (1990); Byrd et al., Psychopharmacology, Vol. 122(2), pp. 95-103 (1995); Byrd et al., Psychopharmacology, Vol. 139(4), pp. 291-299 (1998). Other parameters in blood or urine have examined the formation of carboxyhemaglobin in the blood of smokers and differences in urinary mutagenicity between non-smokers and smokers using their usual cigarette brand or certain other cigarettes. See Doolittle et al., Mutation Research, Vol. 223, pp. 221-232 (1989); Doolittle et al., Food and Chemical Toxicology, Vol. 28, pp. 639-646 (1990); Smith et al., Mutation Research, Vol. 361, pp. 1-9 (1996); Smith et al., Human Exp. Toxicology, Vol. 17(1), pp. 29-34 (1998); Smith et al., Mutation Research, Vol. 470, pp. 53-70 (2000). Urine mutagenicity has also been measure in smokers and non-smokers under various dietary regimens consisting of boiled, baked, or fried foods. See Rahn et al., Environmental and Molecular Mutagenesis, Vol. 17, pp. 244-252 (1991). Studies also have been performed to determine differences between smokers and non-smokers with respect to typical clinical parameters in blood and urine and measures of pulmonary function. See McKarns et al., Modern Pathology, Vol. 8, pp. 434-440 (1995); McKarns et al., Human and Experiemental Toxicology, Vol. 15, pp. 523-532 (1996).

The use of biomarkers is important to assess the exposure of humans to tobacco smoke. See Clearing the Smoke, Institute of Medicine (2001). Various biomarkers can be used to determine exposure of humans to specific compounds found in tobacco smoke. For example, carboxyhemoglobin in blood is an exposure marker for carbon monoxide, urinary mutagenicity is an exposure marker for tobacco smoke mutagens, and nicotine and nicotine metabolites in blood, urine, and saliva are exposure markers for nicotine in tobacco smoke. See Smith et al., Human Exp. Toxicology, Vol. 17(1), pp. 29-34 (1998); Doolittle et al., Mutation Research, Vol. 223, pp. 221-232 (1989); Doolittle et al., Food and Chemical Toxicology, Vol. 28, pp. 639-646 (1990); Smith et al., Mutation Research, Vol. 361, pp. 1-9 (1996); Smith et al., Human Exp. Toxicology, Vol. 17(1), pp. 29-34 (1998); Smith et al., Mutation Research, Vol. 470, pp. 53-70 (2000); Chemical and Biological Studies on New Cigarette Prototypes that Heat Instead of Burn Tobacco, R. J. Reynolds Tobacco Company, 1988; Robinson et al., The Toxicologist, Vol. 8, p. 205 (1988); deBethizy et al., Journal of Clinical Pharmacology, Vol. 30, pp. 755-763 (1990); Byrd et al., Psychopharmacology, Vol. 122(2), pp. 95-103 (1995); Byrd et al., Psychopharmacology, Vol. 139(4), pp. 291-299 (1998). Additionally, certain TSNA and their metabolites, hydroxypropylmercapturic acid, t,t-muconic acid or s-phenylmercapturic acid, and 1-hydroxypyrene levels in urine have been used as exposure markers for TSNA, acrolein, benzene, and polycyclic aromatic hydrocarbons, respectively. See Brown et al., Biomarkers of Tobacco Smoke Exposure: Comparison Between Smokers and Nonsmokers, CORESTA Congress (2002); Byrd and Ogden, Journal of Mass Spectrometry, Vol. 38, pp. 98-107 (2003). Finally, human biomarkers of exposure may include endpoints that examine modifications of macromolecules like DNA and proteins-that result from interactions with tobacco smoke components. Examples would include determination of 8-OhdG DNA adducts in urine and mouthwash samples or carbonyl protein adducts in mouthwash samples. See Brown et al., Biomarkers of Tobacco Smoke Exposure: Comparison Between Smokers and Nonsmokers, CORESTA Congress (2002); Bombick et al., Toxicological Sciences, Vol. 72, S-1 (2003). Biomarkers have been measured from humans smoking cigarettes from the US market or certain other cigarettes. See Chemical and Biological Studies on New Cigarette Prototypes that Heat Instead of Burn Tobacco, R. J. Reynolds Tobacco Company, 1988; Doolittle et al., Mutation Research, Vol. 223, pp. 221-232 (1989); deBethizy et al., Journal of Clinical Pharmacology, Vol. 30, pp. 755-763 (1990); Smith et al., Human Exp. Toxicology, Vol. 17(1), pp. 29-34 (1998); Brown et al., Biomarkers of Tobacco Smoke Exposure: Comparison Between Smokers and Nonsmokers, CORESTA Congress (2002).

The references cited in the paragraphs above are herein incorporated by reference in their entirety.

It would be desirable to test tobacco and tobacco smoke components using more efficient and effective profiling methods which yield reproducible pattern change representative of the entire mixture instead of relying on multiple independent analyses which only examine one chemical, one class of chemicals, or one endpoint at a time.

Embodiments of the present invention relate to tobacco and tobacco compositions, and in particular to the testing of tobacco components, the determination of tobacco component exposure in smokers or substrates exposed to tobacco compositions, and the relationship between tobacco component exposure and the detection of tobacco component-related biological changes in smokers or substrates exposed to tobacco compositions, or the analysis of the chemical constituents of various tobaccos or tobacco compositions and components.

It is therefore an advantage of some, but not necessarily all, embodiments of the present invention to provide methods to analyze substrates, and samples collected from substrates, exposed to tobacco smoke components.

Additional advantages of various embodiments of the invention are set forth, in part, in the description that follows and, in part, will be apparent to one of ordinary skill in the art from the description and/or from the practice of the invention.

### SUMMARY OF THE INVENTION

An innovative analytical method comprising the step of exposing a first substrate to at least one tobacco component, a tobacco composition, or at least one component derived from tobacco, such as, but not limited to, at least one tobacco smoke component. An additional step may comprise the step of preparing a second substrate not exposed to the tobacco component in an identical manner, wherein the second substrate may receive a different exposure, no exposure, or exposure in combination with another factor or condition, or any combination thereof. Additional steps may comprise collecting at least one first sample from the first substrate and at least one second sample from the second substrate, and analyzing the first and second sample by using at least one analytical characterization technique, wherein the analytical characterization technique comprises NMR spectroscopy. In some embodiments, the sample may be the entire substrate or may be limited to certain portions of the substrate, or substances secreted, excreted, or found in or around the substrate. An additional step may comprise comparing the results of the analysis of the first and second samples using data pattern recognition techniques. The step of analyzing the first and second sample may further comprise the use of at least one additional analytical characterization technique. It is preferred that one of the at least one analysis technique is NMR spectroscopy.

The additional analytical characterization techniques may be spectroscopy, chromatography, a combination of spectroscopy and chromatography, mass spectroscopy, gas chromatography, liquid chromatography, and/or ion or ion exchange chromatography, or any other suitable analysis technique.

The tobacco component may be tobacco itself, or any separated and/or isolated portion of the tobacco, or any form of the tobacco. The tobacco smoke component may be obtained from cigarette tobacco and may be collected from smoke generated by a burning cigarette. The tobacco smoke component may be in the form of a tobacco smoke condensate, total tobacco smoke, whole tobacco smoke, and/or tobacco smoke gas phase.

The first and second substrate may comprise a microsomal mixture, metabolic enzyme system, cell, cell culture, tissue, organ, animal, mammal, rodent, and/or human. The at least one first and second sample may be any of the above and in addition may comprise cell culture media, cell lysate, homogenate, biofluid, and/or body fluid. The body fluid may comprise, but is not limited to, blood and urine.

The step of comparing results of the analysis of the first and second samples may be manual and/or automated. The method may further comprise the step of formulating a pattern of chemical constituents or biomarkers present in the sample. The method may also comprise the step of using metabonomic analysis.

An alternative embodiment of the present invention is an analytical method comprising the steps of collecting at least one first tobacco component, collecting at least one second tobacco component, analyzing the first and second tobacco component by using at least one analytical characterization technique, wherein the analytical characterization technique comprises NMR spectroscopy, and comparing results of the analysis of the first and second tobacco component using data pattern recognition techniques. The first and second tobacco component may be whole tobacco, and/or a tobacco smoke component.

It is to be understood that both the foregoing general description and the following detailed description are exemplary and explanatory only, and are not restrictive of the invention as claimed.

### DETAILED DESCRIPTION OF EMBODIMENTS OF THE INVENTION

Reference will now be made in detail to embodiments of the present invention. An embodiment of the present invention is an analytical method comprising the steps of exposing a substrate to tobacco smoke components or compositions, and collecting at least one first sample from the substrate exposed to the tobacco smoke components or compositions. An embodiment may comprise the step of collecting at least one second sample as a reference or control, analyzing the first sample and the second sample by using at least one analytical characterization technique, wherein one of the techniques comprises nuclear magnetic resonance (NMR) spectroscopy, and comparing a result of the analysis of the first sample to a result of the analysis of the second sample. The term control as used herein pertains to a reference substrate or sample that does not receive or contain the identical exposure, condition, or stimulus to that of the first substrate. The absence of the exposure to the test stimulus in the control or reference may be absolute, partial, or may be related to a specific time frame of exposure or non-exposure to the test stimulus. For purpose of illustration, control or reference substrates may be exposure free, or exposure free for a predetermined period before sampling. Samples and/or substrates not subjected to identical exposure conditions may differ with respect to abstinence, use, amounts, and time frames over which the product was used or any other suitable variable or combination. As such, samples obtained from substrates may result in different exposures. In addition, the method according to an embodiment of the present invention may be used to analyze the differences between tobaccos and/or tobacco compositions and components.

The substrates may include, but are not limited to, molecules, metabolites, microsomes, microsomal mixtures, metabolic enzyme systems, cells, cell cultures, tissues, organs, and/or animals. The animals may be mammal or non-mammal. The animals may be, but are not limited to, rabbits, rodents, humans, and those described in Robertson, Donald G., et al., Metabonomic Technology as a Tool for Rapid Throughput In Vivo Toxicity Screening, Comprehensive Toxicology, Vol. xiv, (2002); Application No. PCT/GB01102388, Publication No. WO 01/92880A2; Application No. PCT/GB02/01881, Publication No. WO 02/086478 A2; Application No. PCT/GB02/02758, Publication No. WO 02/099452 A1; application Ser. No. 10/051,615, Publication No. US 2003/0023386 A1; application Ser. No. 10/044,219, Publication No. US 2003/0192707 A1; application Ser. No. 09/945,899, Publication No. US 2002/0136691 A1; Application No. PCT/GB02/01909, Publication No. WO 02/086502 A2; Application No. PCT/GB02/01862, Publication No. WO 02/086501 A2; Application No. PCT/GB02/01854, Publication No. WO 02/086500 A2; Application No. PCT/GB02/01928, Publication No. WO 02/085195 A2; Application No. EP 02/00367, Publication No. WO 02/057989 A2; Application No. PCT/GB01/02559, Publication No. WO 01/96895 A1; and Application No. PCT/GB01/05685, Publication No. WO 02/052293 A1, which are incorporated herein by reference in their entirety.

Depending on the nature of the substrate, a sample collected from the substrate may be comprised of the substrate itself, or may be comprised of a sample collected from the substrate itself, a by-product of the substrate, and/or a component secreted, excreted, or otherwise found in or around the substrate. Samples may include, but are not limited to, cell culture media, cell lysates, organ, tissue, or cell homogenates, or any number of body fluids or biofluids. Body fluids or biofluids may include, but are not limited to, whole blood, milk, saliva, plasma, serum, cerebrospinal fluid (CSF), semen, seminal fluid, aqueous humor, bile, and urine, or any other fluid secreted, excreted, or otherwise derived from a substrate. Samples may also include, but are not limited to, those described in Robertson, Donald G., et al., Metabonomic Technology as a Tool for Rapid Throughput In Vivo Toxicity Screening, Comprehensive Toxicology, Vol. xiv, (2002); Application No. PCT/GB01/02388, Publication No. WO 01/92880 A2; Application No. PCT/GB02/01881, Publication No. WO 02/086478 A2; Application No. PCT/GB02/02758, Publication No. WO 02/099452 A1; application Ser. No. 10/051,615, Publication No. US 2003/0023386 A1; application Ser. No. 10/044,219, Publication No. US 2003/0192707 A1; application Ser. No. 09/945,899, Publication No. US 2002/0136691 A1; Application No. PCT/GB02/01909, Publication No. WO 02/086502 A2; Application No. PCT/GB02/01862, Publication No. WO 02/086501 A2; Application No. PCT/GB02/01854, Publication No. WO 02/086500 A2; Application No. PCT/GB02/01928, Publication No. WO 02/085195 A2; Application No. EP 02/00367, Publication No. WO 02/057989 A2; Application No. PCT/GB00/02559, Publication No. WO 01/96895 A1; and Application No. PCT/GB01/05685, Publication No. WO 02/052293 A1, which are herein incorporated by reference in their entirety.

Tobacco smoke components may include, but are not limited to, total smoke, whole smoke, smoke condensate, and smoke gas phase. Total smoke is defined as that smoke that is received directly from the tobacco product without further processing. Whole smoke is defined as tobacco smoke that is either processed and/or collected, for example, through nitrogen traps. Condensate smoke is defined as the particulate matter collected from tobacco smoke, for example, by passing the smoke through a filter. The filter may be, but is not limited to, a Cambridge filter pad. An electrostatic precipitator may also be used to trap the particulate matter. The filter and/or precipitator traps or collects the particulate matter or condensate, which may be dissolved for application to the substrate. Gas phase smoke comprises the remaining components of tobacco smoke following removal of the particulate matter. Tobacco smoke components may arise from, but are not limited to, the burning, heating, smoking, consumption, or chewing of tobacco, or any other method of release or application of tobacco smoke components.

The method of exposing the substrate to tobacco smoke components is dependent on the nature of the substrate. Substrates may be exposed to tobacco smoke components by application of the component to the surface of the substrate, injection of the component into the substrate, exposure of the substrate to tobacco smoke components, inhalation of the component by the substrate, or any other suitable method. Representative techniques are shown in Coggins, Toxicologic Pathology, Vol. 29, pp. 550-57 (2001); Baumgartner and Coggins, Beitrage zur Tabakforschung International, Vol. 10; pp. 169-174 (1980); Coggins et al., Inhalation Toxicology, Vol. 5(1), pp. 77-95 (1993); Chemical and Biological Studies on New Cigarette Prototypes that Heat Instead of Burn Tobacco, R. J. Reynolds Tobacco Company, 1988; Meckley et al., The Toxicologist, No. 1085 (1998); Meckley et al., The Toxicologist, No. 1107 (1999); Coggins et al., Fundamental Applied Toxicology, Vol. 13, pp. 460-483 (1989); Coggins et al., Inhalation Toxicology, Vol. 1, pp. 197-226 (1989); Doolittle et al., Mutation Research, Vol. 206, pp. 141-148 (1988); Doolittle et al., Mutation Research, Vol. 260, pp. 9-18 (1991); Lee et al., Fundamental & Applied Toxicology, Vol. 19, pp. 141-146 (1992); Brown et al., Experimental and Toxicologic Pathology, Vol. 47, pp. 183-191 (1995); Brown et al., Environmental and Molecular Mutagenesis, Vol. 29, pp. 303-311 (1997); Brown et al., Mutation Research, Vol. 414, pp. 21-30 (1998); Chang et al., Tobacco Science, Vol. 42, pp. 38-45 (1998); Brown et al., Toxicological Sciences, Vol. 47, pp. 33-39 (1999); Lee et al., Mutation Research, Vol. 240, pp. 251-257 (1990); Ayres et al., Inhalation Toxicology, Vol. 7, pp. 1225-1246 (1995); deBethizy et al., Journal of Clinical Pharmacology, Vol. 30, pp. 755-763 (1990); Chang and deBethizy, The Toxicologist, Vol. 36(1), p. 30 (1997); Chemical and Biological Studies on New Cigarette Prototypes that Heat Instead of Burn Tobacco, R. J. Reynolds Tobacco Company, 1988; Pritchard et al., Psychophysiology, Vol. 28 (1991); Pritchard et al., Psychopharmacology, Vol. 108(4), pp. 437-442 (1992); Robinson et al., Psychopharmacology, Vol. 108, pp. 466-472 (1992); Robinson et al., Psychopharmacology, Vol. 108, pp. 466-472 (1992); Pritchard et al., Psychophysiology, Vol. 32, pp. 19-27 (1995); Pritchard et al., Psychopharmacology, Vol. 128, p. 432 (1996); Pritchard et al., Psychopharmacology, Vol. 127, pp. 55-62 (1996); Pritchard et al., Neuropsychobiology, Vol. 34, pp. 208-221 (1996); Walker et al., Assessment of Possible Perceptual, Cognitive and Affective Effects of Sidestream Smoke on Non-Smokers, 1996 CORESTA Congress (1997); Pritchard et al., Psychopharmacology, Vol. 143(3), pp. 273-279 (1999); Stiles et al., The Toxicologist, Vol. 48, pp. 119-120 (1999); Chemical and Biological Studies on New Cigarette Prototypes that Heat Instead of Burn Tobacco, R. J. Reynolds Tobacco Company, 1988; Robinson et al., The Toxicologist, Vol. 8, p. 205 (1988); deBethizy et al., Journal of Clinical Pharmacology, Vol. 30, pp. 755-763 (1990); Byrd et al., Psychopharmacology, Vol. 122(2), pp. 95-103 (1995); Byrd et al., Psychopharmacology, Vol. 139(4), pp. 291-299 (1998); Doolittle et al., Mutation Research, Vol. 223, pp. 221-232 (1989); Doolittle et al., Food and Chemical Toxicology, Vol. 28, pp. 639-646 (1990); Smith et al., Mutation Research, Vol. 361, pp. 1-9 (1996); Smith et al., Human Exp. Toxicology, Vol. 17(1), pp. 29-34 (1998); Smith et al., Mutation Research, Vol. 470, pp. 53-70 (2000); Rahn et al., Environmental and Molecular Mutagenesis, Vol. 17, pp. 244-252 (1991); McKarns et al., Modern Pathology, Vol. 8, pp. 434-440 (1995); McKarns et al., Human and Experiemental Toxicology, Vol. 15, pp. 523-532 (1996), which are herein incorporated by reference in their entirety.

The step of collecting at least one sample from the substrate depends on the nature of the substrate. Collection may occur by aspirating cell culture media, collecting actual cells, tissues, or organs, isolating cell components, such as, but not limited to, microsomes, microsomal mixtures, metabolites, proteins, or by collecting byproducts from the substrates, such as, but not limited to, body fluids or biofluids. Biofluids may include any biological fluid secreted, excreted, or otherwise produced or found in conjunction with an organism. Body fluids may comprise, but are not limited to, blood, plasma, serum, milk, semen, cerebrospinal fluid, urine, or any other suitable body fluid. Commonly used methods and protocols for the collection of samples and substrates are described in Robertson, Donald G., et al., Metabonomic Technology as a Tool for Rapid Throughput In Vivo Toxicity Screening, Comprehensive Toxicology, Vol. xiv, (2002); Application No. PCT/GB01/02388, Publication No. WO 01/92880 A2; Application No. PCT/GB02/01881, Publication No. WO 02/086478 A2; Application No. PCT/GB02/02758, Publication No. WO 02/099452 A1; application Ser. No. 10/051,615, Publication No. US 2003/0023386 A1; application Ser. No. 10/044,219, Publication No. US 2003/0192707 A1; application Ser. No. 09/945,899, Publication No. US 2002/0136691 A1; Application No. PCT/GB02/01909, Publication No. WO 02/086502 A2; Application No. PCT/GB02/01862, Publication No. WO 02/086501 A2; Application No. PCT/GB02/01854, Publication No. WO 02/086500 A2; Application No. PCT/GB02/01928, Publication No. WO 02/085195 A2; Application No. EP 02/00367, Publication No. WO 02/057989 A2; Application No. PCT/GB01/02559, Publication No. WO 01/96895 A1; and Application No. PCT/GB01/05685, Publication No. WO 02/052293 A1, which are herein incorporated by reference in their entirety.

The analysis of the samples, both substrate and control, is performed by nuclear magnetic resonance (NMR) spectroscopy and may include analysis by the additional techniques of mass spectroscopy, or any suitable chromatographic technique, such as, but not limited to, ion, gas, and/or liquid chromatography. In addition to proton (¹H) NMR, other NMR techniques may be used, including, but not limited to, 2-dimensional (2D) NMR methods, COSY (Correlation Spectroscopy), TOCSY (Total Correlation Spectroscopy), inverse-detected heteronuclear correlation methods such as HMBC (Heteronuclear Multiple Bond Correlation), HSQC (Heteronuclear Single Quantum Coherence), and HMQC (Heteronuclear Multiple Quantum Coherence), 2-DJ-Resolved (JRES) methods, spin-echo methods, relaxation edition, diffusion editing, and/or multiple quantum filtering. The analysis may also comprise, in addition to or in combination with NMR spectroscopy, any of the analytical techniques described in Robertson, Donald G., et al., Metabonomic Technology as a Tool for Rapid Throughput In Vivo Toxicity Screening, Comprehensive Toxicology, Vol. xiv, (2002); Application No. PCT/GB01/02388, Publication No. WO 01/92880 A2; Application No. PCT/GB02/01881, Publication No. WO 02/086478 A2; Application No. PCT/GB02/02758, Publication No. WO 02/099452 A1; application Ser. No. 10/051,615, Publication No. US 2003/0023386 A1; application Ser. No. 10/044,219, Publication No. US 2003/0192707 A1; application Ser. No. 09/945,899, Publication No. US 2002/0136691 A1; Application No. PCT/GB02/01909, Publication No. WO 02/086502 A2; Application No. PCT/GB02/01862, Publication No. WO 02/086501 A2; Application No. PCT/GB02/01854, Publication No. WO 02/086500 A2; Application No. PCT/GB02/01928, Publication No. WO 02/085195 A2; Application No. EP 02/00367, Publication No. WO 02/057989 A2; Application No. PCT/GB01/02559, Publication No. WO 01/96895 A1; and Application No. PCT/GB01/05685, Publication No. WO 02/052293 A1, which are herein incorporated by reference in their entirety. If an analytical technique in addition to NMR spectroscopy is employed, that additional technique may be performed before, during, or following the NMR spectroscopy analysis.

The data from the use of these analytical techniques provide a map or fingerprint of the metabolites, molecules, compounds, chemical constituents, etc. present in the sample analyzed. The data may be used to compare differences in the samples, or to compare the samples to known reference standards. Substrates and/or samples may be analyzed and processed for pattern recognition and subjected to statistical analysis using any type of pattern recognition or metabonomic technology, including, but not limited to, the types of technology available from Metabometrix, Ltd. Representative techniques are shown in Clearing the Smoke, Institute of Medicine (2001); Smith et al., Human Exp. Toxicology, Vol. 17(1), pp. 29-34 (1998); Doolittle et al., Mutation Research, Vol. 223, pp. 221-232 (1989); Doolittle et al., Food and Chemical Toxicology, Vol. 28, pp. 639-646 (1990); Smith et al., Mutation Research, Vol. 361, pp. 1-9 (1996); Smith et al., Human Exp. Toxicology, Vol. 17(1), pp. 29-34 (1998); Smith et al., Mutation Research, Vol. 470, pp. 53-70 (2000); Chemical and Biological Studies on New Cigarette Prototypes that Heat Instead of Burn Tobacco, R. J. Reynolds Tobacco Company, 1988; Robinson et al., The Toxicologist, Vol. 8, p. 205 (1988); deBethizy et al., Journal of Clinical Pharmacology, Vol. 30, pp. 755-763 (1990); Byrd et al., Psychopharmacology, Vol. 122(2), pp. 95-103 (1995); Byrd et al., Psychopharmacology, Vol. 139(4), pp. 291-299 (1998); Brown et al., Biomarkers of Tobacco Smoke Exposure: Comparison Between Smokers and Nonsmokers. Byrd and Ogden, Journal of Mass Spectrometry, Vol. 38, pp. 98-107 (2003); Brown et al., Biomarkers of Tobacco Smoke Exposure: Comparison Between Smokers and Nonsmokers, CORESTA Congress (2002); Bombick et al., Toxicological Sciences, Vol. 72, S-1 (2003); Chemical and Biological Studies on New Cigarette Prototypes that Heat Instead of Burn Tobacco, R. J. Reynolds Tobacco Company, 1988; Doolittle et al., Mutation Research, Vol. 223, pp. 221-232 (1989); deBethizy et al., Journal of Clinical Pharmacology, Vol. 30, pp. 755-763 (1990); Smith et al., Human Exp. Toxicology, Vol. 17(1), pp. 29-34 (1998); Brown et al., Biomarkers of Tobacco Smoke Exposure: Comparison Between Smokers and Nonsmokers (2002), which are herein incorporated by reference in their entirety.

Methods and protocols for conducting these analytical techniques are well documented and known to those in the art. Representative techniques for the analytical characterization of the samples, the subsequent analysis of the data, and the procedures available for developing pattern recognition and biomarkers are described in Robertson, Donald G., et al., Metabonomic Technology as a Tool for Rapid Throughput In Vivo Toxicity Screening, Comprehensive Toxicology, Vol. xiv, (2002); Application No. PCT/GB01/02388, Publication No. WO 01/92880 A2; Application No. PCT/GB02/01881, Publication No. WO 02/086478 A2; Application No. PCT/GB02/02758, Publication No. WO 02/099452 A1; application Ser. No. 10/051,615, Publication No. US 2003/0023386 A1; application Ser. No. 10/044,219, Publication No. US 2003/0192707 A1; application Ser. No. 09/945,899, Publication No. US 2002/0136691 A1; Application No. PCT/GB02/01909, Publication No. WO 02/086502 A2; Application No. PCT/GB02/01862, Publication No. WO 02/086501 A2; Application No. PCT/GB02/01854, Publication No. WO 02/086500 A2; Application No. PCT/GB02/01928, Publication No. WO 02/085195 A2; Application No. EP 02/00367, Publication No. WO 02/057989 A2; Application No. PCT/GB01/02559, Publication No. WO 01/96895 A1; and Application No. PCT/GB01/05685, Publication No. WO 02/052293 A1, which are incorporated herein in their entirety by reference.

The step of comparing the results of the analysis of the substrate and control samples may be performed manually by visual examination, or may be compared by automated means, including, but not limited to, scanning, computer analysis, pattern recognition analysis, or any other suitable means. Substrates and/or samples may be analyzed and processed for pattern recognition and subjected to statistical analysis using any type of metabonomic technology, including, but not limited to, the types of technology available from Metabometrix, Ltd. Metabonomic technology involves the use of an analytical technique, such as NMR spectroscopy and various types of pattern recognition technologies to evaluate the metabolic characteristics of an animal from samples obtained from that animal. See Robertson, Donald G., et al., Metabonomic Technology as a Tool for Rapid Throughput In Vivo Toxicity Screening, Comprehensive Toxicology, Vol. xiv, 2002. Metabonomic technologies may also be used to evaluate the differences in chemical compositions and constituents within various samples. The samples need not have been exposed to a biological system to perform this analysis. Pattern recognition technology, independent or in conjunction with metabonomics, can be used to identify patterns of data resulting from sample analysis. The presence of these patterns may obviate the need to identify each individual chemical constituent or metabolite present in an analyzed sample. Pattern recognition analysis includes, but is not limited to, techniques such as principal components analysis, both unsupervised and supervised, and are discussed in the references cited below. Pattern recognition technology, including the use of metabonomics, provides for the possible generation of biomarkers. Biomarkers may be identified if a pattern can be reproducibly associated with similar or similarly treated samples. Methods and protocols for the comparison of the results are described in Robertson, Donald G., et al., Metabonomic Technology as a Tool for Rapid Throughput In Vivo Toxicity Screening, Comprehensive Toxicology, Vol. xiv, (2002); Application No. PCT/GB01/02388, Publication No. WO 01/92880 A2; Application No. PCT/GB02/01881, Publication No. WO 02/086478 A2; Application No. PCT/GB02/02758, Publication No. WO 02/099452 A1; application Ser. No. 10/051,615, Publication No. US 2003/0023386 A1; application Ser. No. 10/044,219, Publication No. US 2003/0192707 A1; Application No. 09/945,899. Publication No. US 2002/0136691 A1; Application No. PCT/GB02/01909, Publication No. WO 02/086502 A2; Application No. PCT/GB02/01862, Publication No. WO 02/086501 A2; Application No. PCT/GB02/01854, Publication No. WO 02/086500 A2; Application No. PCT/GB02/01928, Publication No. WO 02/085195 A2; Application No. EP 02/00367, Publication No. WO 02/057989 A2; Application No. PCT/GB01/02559, Publication No. WO 01/96895 A1; and Application No. PCT/GB01/05685, Publication No. WO 02/052293 A1, which are herein incorporated in their entirety by reference.

In another embodiment of the present invention, the method further comprises the step of formulating a pattern of biomarkers present in the substrate exposed to the tobacco smoke components. Biomarkers may be formulated by the process of pattern recognition within an NMR spectruma. A discussion of the formulation of biomarkers and recognition of biomarker patterns are described in Robertson, Donald G., et al., Metabonomic Technology as a Tool for Rapid Throughput In Vivo Toxicity Screening, Comprehensive Toxicology, Vol. xiv, (2002); Application No. PCT/GB01/02388, Publication No. WO 01/92880 A2; Application No. PCT/GB02/01881, Publication No. WO 02/086478 A2; Application No. PCT/GB02/02758, Publication No. WO 02/099452 A1; application Ser. No. 10/051,615, Publication No. US 2003/0023386 A1; application Ser. No. 10/044,219, Publication No. US 2003/0192707 A1; application Ser. No. 09/945,899, Publication No. US 2002/0136691 A1; Application No. PCT/GB02/01909, Publication No. WO 02/086502 A2; Application No. PCT/GB02/01862, Publication No. WO 02/086501 A2; Application No. PCT/GB02/01854, Publication No. WO 02/086500 A2; Application No. PCT/GB02/01928, Publication No. WO 02/085195 A2; Application No. EP 02/00367, Publication No. WO 02/057989 A2; Application No. PCT/GB01/02559, Publication No. WO 01/96895 A1; and Application No. PCT/GB01/05685, Publication No. WO 02/052293 A1, which are herein incorporated in their entirety by reference.

The methods discussed above may be applied to the analysis of substrates and/or samples in the context of analyzing differences in biological effect of tobacco compositions and components and/or chemical composition and constituents of tobaccos by using NMR spectroscopy, and other analytical methods. The analysis may be used to determine the differences between many conditions, including, but not limited to the differences in smoking versus non-smoking humans or differences in substrates exposed to tobacco compositions under varying conditions. Metabonomic analysis may be used. Novel biomarkers or other patterns may be identified. An embodiment of the present invention permits the multi-dimensional and non-invasive analysis, for example, of smokers versus non-smokers. The results of differences across broad spectrums in two or more samples may be addressed. The effect of many factors, such as, but not limited to, age, alcohol consumption, diet, genetic predisposition, and/or disease permits a multi-variant analysis with any number of samples.

In an alternative method according to an embodiment of the present invention, the method can be used to compare the chemistries and/or chemical constituents of two or more tobaccos, compositions and/or components derived from tobaccos. The analysis may involve the tobacco, composition, and components themselves or compositions and components derived from tobacco smoke. This analysis could include, but is not limited to, the analysis of various types of tobaccos, blends of tobaccos, various tobacco treatments, and/or various processing methods, such as, but not limited to, how the tobacco is grown, harvested, cured, aged, and/or stored. The processes may include, but are not limited to, reconstitution processes, expansion processes, and heat treatments. The effects of various tobacco additives on the tobacco itself may be investigated. Casing and top dressing components, such as flavors, as well as the effect of certain preservatives, or insecticides or pesticides used during the growth of the tobacco may be evaluated. The results of these analyses may be used to advance product design, such as, but not limited to, cigarette design, configuration, cigarette papers, filters, air diluted blends, and various tobaccos.

It will be apparent to those skilled in the art that variations and modifications of the present invention can be made without departing from the scope or spirit of the invention. For example, the methods according to embodiments of the present invention include, but are not limited to, methods for the analysis by NMR spectroscopy alone, or in addition to other spectroscopic and chromatographic techniques, of body fluids collected from animals exposed to cigarette smoke components, body organs, tissues, or cells from animals exposed to cigarette smoke components, body fluids from human smokers and non-smokers, body fluids from human smokers before and after smoking cessation, body fluids from human smokers before and after smoking potentially reduced exposure cigarettes, body fluids from human smokers and non-smokers on a controlled dietary regimen, body fluids from human smokers and non-smokers on a variety of dietary regimens, body fluids from asymptomatic smokers and smokers with chronic ailments, including, but not limited to, bronchitis or other pulmonary ailments, and body fluids, cellular components, cells, tissue, organs, or any other appropriate sample, from human smokers and non-smokers, and subsequent analysis of the results.

What is claimed is:

1. An analytical method comprising the steps of:
exposing a first substrate to at least one tobacco component;
preparing a second substrate not exposed to the tobacco component in an identical manner as the first substrate;
collecting at least one first sample from the first substrate and at least one second sample from the second substrate;
analyzing the first and second sample by using at least one analytical characterization technique, wherein the analytical characterization technique comprises NMR spectroscopy; and
comparing results of the analysis of the first and second samples using data pattern recognition techniques.

2. The method according to claim 1, wherein the step of analyzing the first and second sample further comprises the use of at least one additional analytical characterization technique.

3. The method according to claim 1, wherein the tobacco component is from cigarette tobacco.

4. The method according to claim 1, wherein the tobacco component is a tobacco smoke component.

5. The method according to claim 1, wherein the tobacco component is in the form of a tobacco smoke condensate.

6. The method according to claim 1, wherein the tobacco component is in the form of total tobacco smoke.

7. The method according to claim 1, wherein the tobacco component is in the form of whole tobacco smoke.

8. The method according to claim 1, wherein the tobacco component is in the form of tobacco smoke gas phase.

9. The method according to claim 1, wherein the first and second substrate is a microsomal mixture.

10. The method according to claim 1, wherein the first and second substrate is a metabolic enzyme system.

11. The method according to claim 1, wherein the first and second substrate is a cell.

12. The method according to claim 1, wherein the first and second substrate is a cell culture.

13. The method according to claim 1, wherein the first and second substrate is a tissue.

14. The method according to claim 1, wherein the first and second substrate is an organ.

15. The method according to claim 1, wherein the first and second substrate is an animal.

16. The method according to claim 1, wherein the first and second substrate is a mammal.

17. The method according to claim 1, wherein the first and second substrate is a rodent.

18. The method according to claim 1, wherein the substrate is human.

19. The method according to claim 1, wherein the at least one first and second sample is cell culture media.

20. The method according to claim 1, wherein the at least one first and second sample is a cell lysate.

21. The method according to claim 1, wherein the at least one first and second sample is a homogenate.

22. The method according to claim 1, wherein the at least one first and second sample is a biofluid.

23. The method according to claim 1, wherein the at least one first and second sample is a body fluid.

24. The method according to claim 23, wherein the body fluid is urine.

25. The method according to claim 23, wherein the body fluid is blood.

26. The method according to claim 2, wherein the additional analytical characterization technique is spectroscopy.

27. The method according to claim 2, wherein the additional analytical characterization technique is chromatography.

28. The method according to claim 2, wherein the additional analytical characterization technique is a combination of spectroscopy and chromatography.

29. The method according to claim 2, wherein the additional analytical characterization technique is mass spectroscopy.

30. The method according to claim 2, wherein the additional analytical characterization technique is gas chromatography.

31. The method according to claim 2, wherein the additional analytical characterization technique is liquid chromatography.

32. The method according to claim 2, wherein the additional analytical characterization technique is ion exchange chromatography.

33. The method according to claim 1, wherein the step of comparing results of the analysis of the first and second samples is automated.

34. The method according to claim 1, further comprising the step of formulating a pattern of biomarkers present in the first sample.

35. An analytical method comprising the steps of:
exposing a first substrate to at least one tobacco smoke component;
preparing a second substrate not exposed to the tobacco smoke component in an identical manner as the first substrate;
collecting at least one first sample from the first substrate and at least one second sample from the second substrate;
analyzing the first and second sample by using at least one analytical characterization technique, wherein the analytical characterization technique comprises NMR spectroscopy; and
comparing results of the analysis of the first and second samples using data pattern recognition techniques.

36. The method according to claim 35, wherein the step of exposing the first substrate to the tobacco smoke component occurs within an animal.

37. The method according to claim 35, wherein the step of exposing the first substrate to the tobacco smoke component occurs within a cell culture.

38. The method according to claim 35, wherein the at least one first and second sample is a biofluid.

39. The method according to claim 35, wherein the at least one first and second sample is a microsomal mixture, metabolic enzyme system, cell, cell culture, tissue, or organ.

40. An analytical method comprising the steps of:
exposing a first substrate to at least one tobacco component;
preparing a second substrate not exposed to the tobacco component in an identical manner as the first substrate;
collecting at least one first sample from the first substrate and at least one second sample from the second substrate;
analyzing the first and second sample by using at least one analytical characterization technique, wherein the analytical characterization technique comprises NMR spectroscopy; and
comparing results of the analysis of the first and second samples using metabonomic data pattern recognition techniques.

41. The method according to claim 40, further comprising the step of formulating a pattern of biomarkers present in the first sample.

42. An analytical method comprising the steps of:
exposing a first substrate to at least one tobacco component;
collecting at least one sample from the first substrate;
analyzing the sample by using at least one analytical characterization technique, wherein the analytical characterization technique comprises NMR spectroscopy, and
comparing results of the analysis of the sample to a reference using data pattern recognition techniques.

43. The method according to claim 42, further comprising the steps of:
preparing a second substrate not exposed to the tobacco component in an identical manner as the first substrate;
collecting at least one second sample from the second substrate;
analyzing the second sample by using at least one analytical characterization technique, wherein the analytical characterization technique comprises NMR spectroscopy; and
comparing results of the analysis of the second sample using data pattern recognition techniques.

44. An analytical method comprising the steps of:
collecting at least one first tobacco component;
collecting at least one second tobacco component;
analyzing the first and second tobacco component by using at least one analytical characterization technique, wherein the analytical characterization technique comprises NMR spectroscopy; and
comparing results of the analysis of the first and second tobacco component using data pattern recognition techniques.

45. The method according to claim 44, wherein the first and second tobacco component is whole tobacco.

46. The method according to claim 44, wherein the first and second tobacco component is a tobacco smoke component.

47.
